(19) United States
(12) Patent Application Publication
(10) Pub. No.: US 2023/0268061 A1
(43) Pub. Date: Aug. 24, 2023

(54) Systems and Methods to Track and Automate Home Care Management

(71) Applicant: MICHAEL ASHY, Dallas, TX (US)

(72) Inventor: MICHAEL ASHY, Dallas, TX (US)

(21) Appl. No.: 17/677,276

(22) Filed: Feb. 22, 2022

(51) Int. Cl. G16H 40/20; G16H 10/60; G16H 40/67; G06Q 10/06

(52) U.S. Cl. CPC G16H 40/20; G16H 10/60; G16H 40/67; G06Q 10/063114

(57) ABSTRACT

Home care management tasks may be tracked, scheduled, verified, and accounted for using devices interacting with each other to maintain an overall “care plan” for a given care recipient (“CR”). The CR represents a patient in need of home health services (or other support services) that may be provided by a group of actors connected virtually through a computer managed network providing a care management system. The care management system includes logic and devices that interact to create an automated care plan. Data underlying the care plan is secure and protected (e.g., follows HIPAA rules). A matrix of access permissions is maintained based on actors, roles of actors, and type of data maintained. Actors may be family members, volunteers, medical professionals, or other service providers. Location data for an actor may determine when or if an alert style notification is sent to a device associated with that actor.

### BACKGROUND

Home care management will affect most families later in their lives. As a parent ages, adult children and other family members may be responsible for care to assist the aged parent. In some cases, an accident or illness may result in a younger person receiving similar assistance. In either case, providing home based assistance is a complicated, time-consuming, emotional, and expensive proposition.

Historically, providing home care has been a centralized process where a care recipient (the person in need of the care) typically has one or two primary care coordinators (usually an adult child or spouse) that manage and maintain a list of needs (tasks) to be performed for the care recipient (“CR”). A CR may also be referred to as a patient.

Tasks need to be coordinated and tracked to ensure that the CR is receiving an appropriate level of care. The types of tasks and level of care may change over time as the health status of the CR either improves or declines. There also may be instances where instantaneous urgent care is needed, such as a fall for an elderly person, or a sudden change in condition (blood sugar level, heart condition, etc.).

There is a need to address the above concerns, automate scheduling and notification regarding tasks and information, and improve the home care management process. Accordingly, systems, methods, and devices disclosed herein provide a collaborative networking capability (conceptually a functional social networking capability) for a collaborative system (of devices, users, providers) that is distributed and securely controlled to assist in providing home health care for a target care recipient. Multiple actors (providers of service or support) may interact with the care recipient and results of that interaction (e.g., completion of a task) may be shared throughout the disclosed automated, secure, and hierarchical information system underpinning a collaborative set of devices and users.

### BRIEF DESCRIPTION OF THE DRAWINGS

For a detailed understanding of various examples, accompanying drawings are provided. The following figures form part of the present specification and are included to further demonstrate certain aspects of the present claimed subject matter. These figures are examples that are not necessarily drawn to scale and should not be used to limit or restrict the present claimed subject matter. The present claimed subject matter may be better understood by reference to one or more of these drawings in combination with the description of embodiments presented herein. Consequently, a more complete understanding of the present embodiments and further features and advantages thereof may be acquired by referring to the following description taken in conjunction with the accompanying drawings. Additionally, like reference numerals in the drawings identify identical or substantially similar elements, wherein:

FIG. 1 is an example of a high-level architectural and functional diagram for a home care management system according to one or more examples of the disclosure;

FIG. 2 is an example of a care plan that outlines tasks (e.g., in the form of a daily schedule) and needs for a CR according to one or more examples of this disclosure;

FIG. 3 is an example of different phases of mobile device setup such that the mobile device may interact with an automated care management system, according to one or more examples of this disclosure;

FIG. 4 is an example of a home care management interface for use by an actor (e.g., role of patient or family member) to assist in information gathering regarding a particular CR according to one or more examples of this disclosure;

FIG. 5 is an example of a first set of tabular representations of support data that may be managed within a care management system for a particular CR according to one or more examples of this disclosure;

FIG. 6 is an example of a second set of tabular representations of data (e.g., attributes of a particular CR) that may also be managed within a care management system for a particular CR according to one or more examples of this disclosure;

FIG. 7 is an example of different devices that may be associated with either a CR or an actor where each of these devices may automatically interface with a care management system according to one or more examples of this disclosure;

FIG. 8 is an example of a daily task list (e.g., in the form of a table) for a particular CR and illustrates how that daily task list may be presented on different end user devices as part of a care management system according to one or more examples of this disclosure;

FIG. 9 is a block diagram illustrating interactions of potential actors (each with different roles) and their interaction possibilities with a particular CR utilizing a care management system according to one or more examples of this disclosure;

FIG. 10 is an example of a possible workflow to provide definitional information for a particular CR to set that CR up within a care management system according to one or more examples of this disclosure;

FIG. 11 is an example workflow to provide an action phase (e.g., automated care plan management) for a care management system according to one or more examples of this disclosure;

FIG. 12 is a block diagram of possible device interactions and notes that a single actor may utilize multiple devices associated to a particular CR when that CR is being provided capability of a care management system according to one or more examples of this disclosure;

FIG. 13 is an example of a computing device, including a computer readable medium, that may be used to implement one or more of the workflows (in this case the workflow of FIG. 10 or 11) of this disclosure according to one or more examples of this disclosure;

FIG. 14 is an example of a network infrastructure including networked computer components and devices to illustrate possible processing resources, user interface points, and data pathways, according to one or more examples of this disclosure; and

FIG. 15 is a block diagram providing an example of a computing device that may be used within one or more of the devices shown in FIG. 14 (or even other devices).

### DETAILED DESCRIPTION

Certain terms are used throughout the following description and claims to refer to particular components, configurations of components, and functions provided by people/service providers/computers/networks. As one skilled in the art will appreciate, the same component may be referred to by different names. This document does not intend to distinguish between components that differ in name but not function. In the following discussion and in the claims, the terms “including” and “comprising” are used in an open-ended fashion, and thus should be interpreted to mean “including, but not limited to . . . .”

In the following description, for purposes of explanation, numerous specific details are set forth in order to provide a thorough understanding of the embodiments disclosed herein. It will be apparent, however, to one skilled in the art that the disclosed embodiments may be practiced without these specific details. In other instances, structure and devices are shown in block diagram form to avoid obscuring the disclosed embodiments. Moreover, the language used in this disclosure has been principally selected for readability and instructional purposes and may not have been selected to delineate or circumscribe the inventive subject matter, resorting to the claims being necessary to determine such inventive subject matter. Reference in the specification to “one embodiment” or to “an embodiment” means that a particular feature, structure, or characteristic described in connection with the embodiments is included in at least one embodiment.

The scope of the protection sought herein is defined by the appended claims and equivalents thereof. Illustrative embodiments are described below. In the interest of clarity, not all features of an actual implementation are described in every embodiment disclosed in this specification. In the development of any such actual embodiment, numerous implementation-specific decisions may need to be made to achieve the design-specific goals, which may vary from one implementation to another. It will be appreciated that such a development effort, while possibly complex and time-consuming, would nevertheless be a routine undertaking for persons of ordinary skill in the art having the benefit of this disclosure. As understood by those skilled in the art, elements in flow charts may be performed in the order shown or may be performed in an order different than shown to achieve the same or a similar result.

This disclosure provides for a system to support home care management for a care recipient (referred to herein as a “CR” or possibly a patient) where tasks for homecare management for the CR are performed, scheduled, tracked, and audited by a distributed set of computing devices. The distributed set of computing devices may be considered to create a smart “social network” of parties (referred to herein as “Actors”) that together will provide support for the CR. This smart social network may also be considered a collaborative platform where important task related information is shared as opposed to the kind of information that typically may be shared on a typical social network. Support tasks may include both medical tasks and other tasks for the CR to maintain an acceptable standard of living relative to pre-defined parameters for that CR. In most cases, the pre-defined parameters reflect a goal of maintaining the same standard of living for which the CR was previously accustomed.

Disclosed herein is a centralized, virtualized, caregiving platform predicated on replacing typical care plan systems that are paper based and manually maintained. In the disclosed platform (or system), information may be shared to multiple participants associated with a CR and that information may be shared in real-time or near real-time. Additionally, data mining and other artificial intelligence techniques may be utilized to analyze and adjust care plans automatically.

Using the collaborative network, actors or participants may perform actions to assist a CR. The actions may be scheduled and managed through actor interaction with end devices communicatively coupled to the collaborative network system. Medical devices may provide patient metrics automatically. Care task lists may be generated and tracked for completion. Alerts may be generated to inform actors of non-completed tasks or for urgent care needs. Information may be securely managed and provided in a hierarchical manner to appropriate actors. Actors may include local and remote family members, friends, volunteers, health care providers, and smart devices (e.g., actor devices may be devices that complete tasks such as an automatic medicine dispenser and thus actor devices are different than user devices).

Information sharing may be controlled based on permissions and roles associated with different participants. In this disclosure, actors may be referred to as participants that perform actual care based tasks whereas the term “participants” may include passive entities that are provided information as opposed to being assigned tasks to complete. The difference between participants and actors is subtle and status of an entity enrolled within the disclosed system (e.g., user) may change over time. Accordingly, the terms may be considered interchangeable, and any distinction may be derived from the context in which each term is used.

For example, a remote family member (when located remotely) may be a participant with respect to most of their interaction with the system. However, that remote family member may visit the CR and, during the visit, they may be considered an actor (e.g., because they are local and performing tasks). Further, some tasks in a care plan may be able to be completed by a participant when they are remote so in that case, the remote participant may be considered as performing an action (e.g., is an actor).

A care plan refers historically to paper documents that home health, hospitals, hospice, home care agencies, and the like, utilize to provide services for a CR. In the disclosed system, the care plan is maintained digitally and may be automatically updated by human data entry through a data portal (e.g., a user interface of an application) or may be updated via communication with a medical device taking readings of the CR. In this context, the medical device may be considered an Internet of Things (“IoT”) device. Because the device is an IoT device, it may, in some cases, be remotely controlled or accessed and may be network connected to provide information as necessary to the disclosed system.

As explained further below, the disclosed system receives definitional information about a new CR. This definitional information may be provided by a family member signing up their parent (as an example) who needs home health care. The definitional information represents a starting point and further information about the CR will be provided over time. The definitional information may include information about the health history of the CR and outline a level of care desired for the CR. Again, as time passes the level of care desired may change and the health history will naturally evolve. The disclosed system incorporates these changes over time and automatically adjusts a generated daily task list for the CR to accommodate current needs and desires.

In the disclosed system, different “levels” of information may be collected. For example, information that some actors may consider trivial (e.g., “how did breakfast go today?”) may be collected. Other information like an actual blood pressure reading taken in the morning may be considered less trivial. Specifically, actors/participants may be provided information by using a role for the user as a filter relative to the importance associated with different data inputs. Data inputs may have a type that identifies what kind of data is being input and may have an associated importance level that identifies how significant the data item is. In general, the system may assign many different kinds of attributes to data inputs to assist in filtering the “correct” information to be provided to the “interested” participants. Accordingly, participants of the system may receive appropriate “push” notifications if information obtained is determined to be “significant” to that participant.

In addition to data input types and levels of significance, data thresholds may be pre-defined for data inputs. These data thresholds may be used to alter the level of significance for a specific data input. As a specific example, thresholds for blood pressure readings may be pre-defined such that a doctor (or other participant) is automatically notified when a specific blood pressure reading does not satisfy the threshold conditions. Other collected metrics may have one or more pre-defined thresholds that may be used to initiate alert conditions and set a priority of the alert condition. For example, a determination that a CR has fallen may be reported and several actors may be automatically notified (in near real-time) to address the situation. Once the situation is addressed, the actor that addressed the condition may provide additional information (e.g., through an application user interface) so that all other participants are informed.

In addition to instantaneous reporting of attributes collected relative to a CR, periodic reports may be generated. These reports may be provided to “interested” parties as discussed above (e.g., filtered based on parameters). Using these periodic reports, family members and friends may be kept abreast of the condition of the CR.

The disclosed system may run algorithms (e.g., artificial intelligence algorithms) to analyze all comorbidities of a CR. The comorbidities may be analyzed relative to collected metric readings (e.g., from the IoT medical devices) and the system may utilize a determination by the algorithms to automatically adjust a care task plan for the next day. In this context, the algorithms may provide an extra level of care in addition to that provided by the medical professionals associated with the system and the CR. In some implementations, the behind the scenes algorithms may be considered as a virtual doctor that provides information based on data mining everything known about the CR. As technology evolves, this virtual doctor may be considered an important participant that provides input to the care of a given CR.

As examples, the disclosed system may perform vitals monitoring and provide notifications based on metrics determined during the measuring of a medical “vital.” Some vitals that may be monitored include, but are not limited to, blood pressure, pulse, oxygen saturation, blood sugar, respiration rate, and temperature. In general, any metric measured by a medical device may have thresholds defined relative to a particular CRs history and condition such that customized notification for metrics that are “out of bound” for that CR will trigger one or more notifications.

One goal of the disclosed system may be to reduce or eliminate a “hospital readmission” for the CR. In some cases, comorbidities for a given CR may be a factor that will increase a hospital readmission for that CR. Hospital readmissions increase cost and decrease a CR's quality of life. In general, if comorbidities are taken into account when creating a task care plan for the CR an overall improvement in the quality of life for that CR may be realized. The disclosed system addresses this concern and recognizes how a proper and dynamic task care plan may be used to minimize impact of different comorbidities. Comorbidities may include, for example, congestive heart failure (CHF), chronic obstructive pulmonary disease (COPD), diabetes, atrial fabulation (AFIB), hypertension, kidney disease, liver disease, and cognitive disease.

In addition to chronic diseases, temporary diseases may indicate that an alteration to a task care plan may be desired for a period of time. For example, if a CR has an episode of a stroke or heart attack, some tasks may be implemented to react to that episode. Also, if a CR has a virus or other temporary illness, a specific treatment plan for that illness may be implemented until that temporary illness has been addressed. After the temporary illness has been addressed, tasks associated with that temporary condition may be automatically reduced or eliminated from the automatically generated daily task care plan.

As discussed herein, an initial assessment of a CR may be performed as that CR is introduced (definitional phase) into the disclosed system. Part of the Assessment involves disclosures of conditions and history of the person receiving care. After initial setup for a CR, the disclosed system will collect various scores and data (e.g., in the form of the above-mentioned metrics). Over time, a medical condition of the CR will continue to get updated and may, for example, result in comorbidity numbers increasing. Accordingly, a CR assessment may also dynamically change. This dynamic change may result in a new “suggested” assessment that may get automatically generated and, using the notification aspects of the disclosed system, cause appropriate case managers, family members, and stakeholders to provide a review and approval for changes in care for the CR.

As described throughout this disclosure, the disclosed system provides a real time daily record of medical information, tasks completed around the home, and other information so that all users of the system that are associated with the CR may obtain (or be notified automatically) of information significant to the respective user. The information may be closely controlled to conform to regulations regarding distribution of certain medical information and be controlled so that users are not provided information for which they don't care to receive. Thus, the integrated system disclosed herein can improve the care of a CR, reduce stress on family members, prolong life, improve quality of life, and provide potential lifesaving alerts.

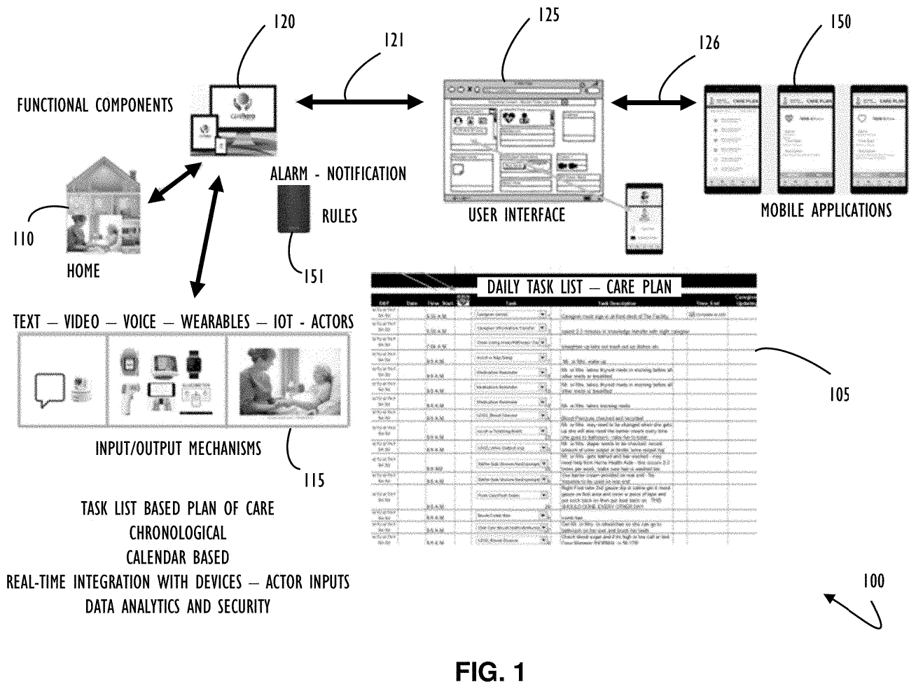

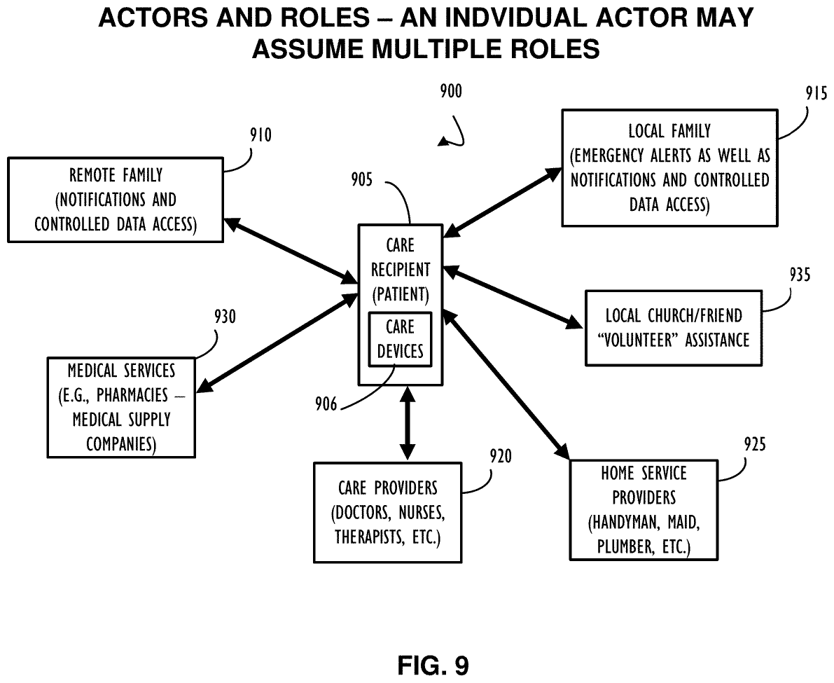

Turning now to the drawings, a description of the architecture, devices, roles, and actors that may participate in providing care for a CR within the disclosed care management system that may be implemented as a cloud-based application will be presented. FIGS. 1-8 provide illustrations including tables of information, devices, and possible informational exchange paths between devices and include screen shots as examples of how actors may interface with the care management system. FIGS. 9-15 provide block diagrams and flow charts to further explain the interactions, flows, and devices that may be used to collect, correlate, and manage the information and interactions that were discussed for FIGS. 1-8 and previously in this disclosure.

Referring now to FIG. 1, an example of functional components 100 for the disclosed home care management system is illustrated. A daily task list in tabular form is illustrated as care plan 105. Care plan 105 is central to the system and represents detailed information regarding how actors are to perform actions for a CR. Care plan 105, as discussed in more detail below, may be automatically generated and maintained to provide a level of care for a CR under home care (e.g., at home 110, as illustrated).

Devices 115 represent input/output mechanisms for the disclosed care management system. Devices 115 may include Internet of Things (“IOT”) devices and communicate data that includes text, video, voice, metrics or other types of digital information. Devices 115 may provide an interface for actors (e.g., people or devices performing tasks) such that their information and notes may be integrated within the care management system.

Home system 120 may provide a communication gateway for all devices 115 within home 110. Arrow 121 indicates that a user interface 125 may be implemented on one or more devices to assist with human interaction to the disclosed care management system. Arrow 126 indicates that mobile applications 150 may also be integrated as a form of user interface 125. Block 151 indicates that alarm and notification rules may be implemented, for example, within home system 120.

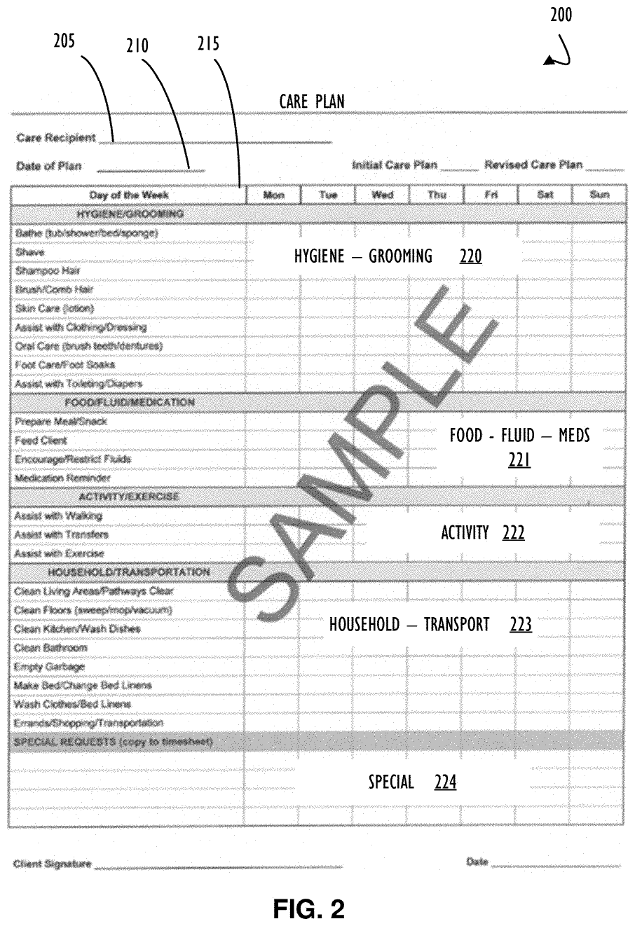

Referring now to FIG. 2, care plan 200 illustrates one example of information that may be included in an automated care plan (e.g., another example of care plan 105 discussed above). Care plan 200 is unique for a particular care recipient (“CR”) and that CR may be identified within care plan 200 as indicated by field 205. Each care plan 200 in this particular example is chronological in nature and maintains schedules of tasks so the date of care plan 200 may be indicated by field 210. Day of week field 215 provides for current and near term future tasks to be coordinated.

As illustrated in care plan 200, information maintained may be of different types. Hygiene-grooming 220 information may be maintained to assist in cleanliness of the CR. Food, fluid, and medications (“meds”), 221 may be provided and monitored as tasks within care plan 200. Activity 222 indicates that exercise and mobility activities may be a part of care plan 200 to maintain physical strength of a given CR. Household-Transport 223 represents another set of tasks that may be maintained within care plan 200 and Special 224 section represents a field to incorporate needs that may not fit into other pre-defined categories of information but are important to provide for the CR.

In general, care plan 200 includes information to provide a level of care and level of living consistent with needs of a particular CR. Care plan 200 is customized to the medical needs of the CR and will automatically adapt over time as those needs change. Similarly, as the CR either improves or degrades, activates that are not specifically health care may be scheduled, monitored, and managed. The disclosed care management system will therefore help ensure activities are performed. Ensuring these activities are monitored and performed in a timely manner may both extend the life of the CR and enhance the remaining life of the CR.

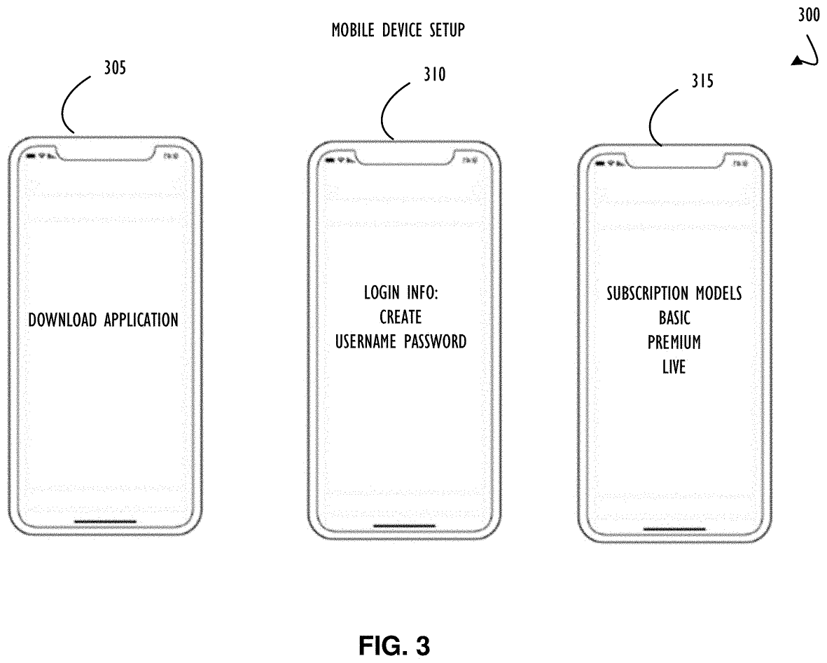

Referring now to FIG. 3, diagram 300 illustrates different phases of setup for a mobile device that may be utilized by an actor to interface with the disclosed care management system. For example, diagram 300 illustrates a possible setup of mobile applications 150 from FIG. 1. At phase 305 the application may be downloaded. This action may be performed by all actors that wish to participate in care plan management for a given CR in the context of the disclosed automated care plan 200 and its interactions/control of the disclosed care management system.

After downloading the application, phase 310 indicates that authentication may be setup by each particular actor. As described throughout this disclosure, each actor may assume one or more roles. Tasks are allocated based on roles and access to information may also be controlled based on roles. The login information allows for security and authentication as would be expected for controlled access such as that discussed herein.

Phase 315 indicates that different subscription models may be provided to determine functionality accessible to different actors. In the illustrated embodiment, there are three subscription models shown—“basic”, “premium”, and “live”. Because different types of actors may have different levels of interaction with the care management system, different subscription models may assist in providing the appropriate level of functionality based on their needs. Each level of access may have an associated different level of cost for the actor/user.

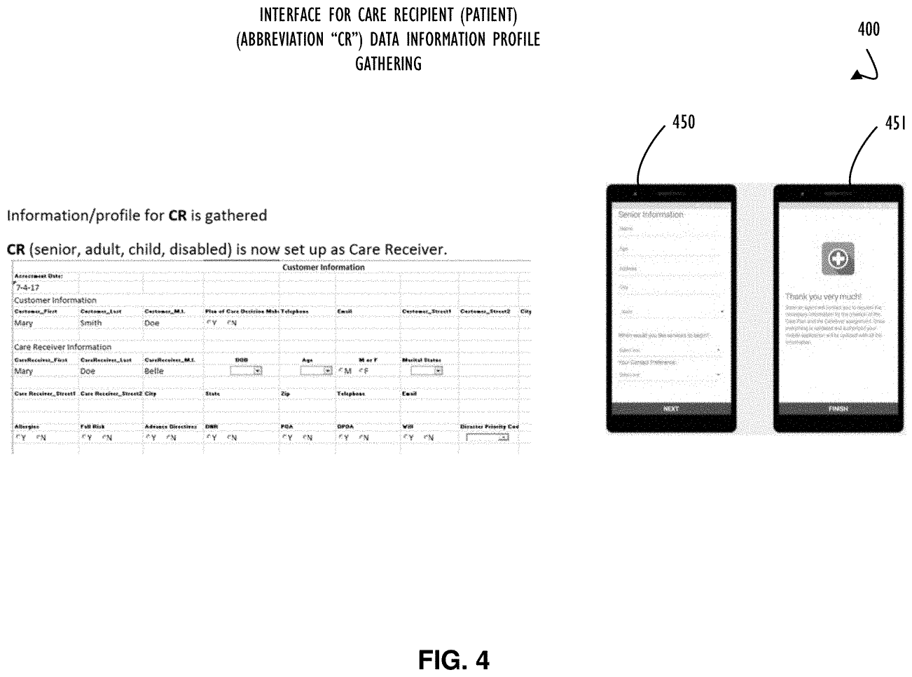

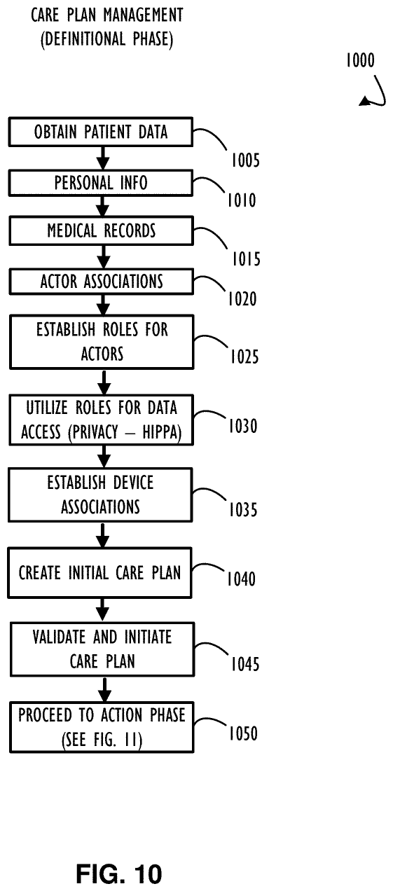

Referring now to FIG. 4, an example of a home care management interface 400 for use by an actor (e.g., in a role of patient or family member) to assist in information gathering regarding a particular CR is illustrated. Interface 400 may be presented as a mobile application interface 450 and 451, and be used to gather definitional information about a CR. In this manner, a CR may be setup within the disclosed care management system (See FIG. 10).

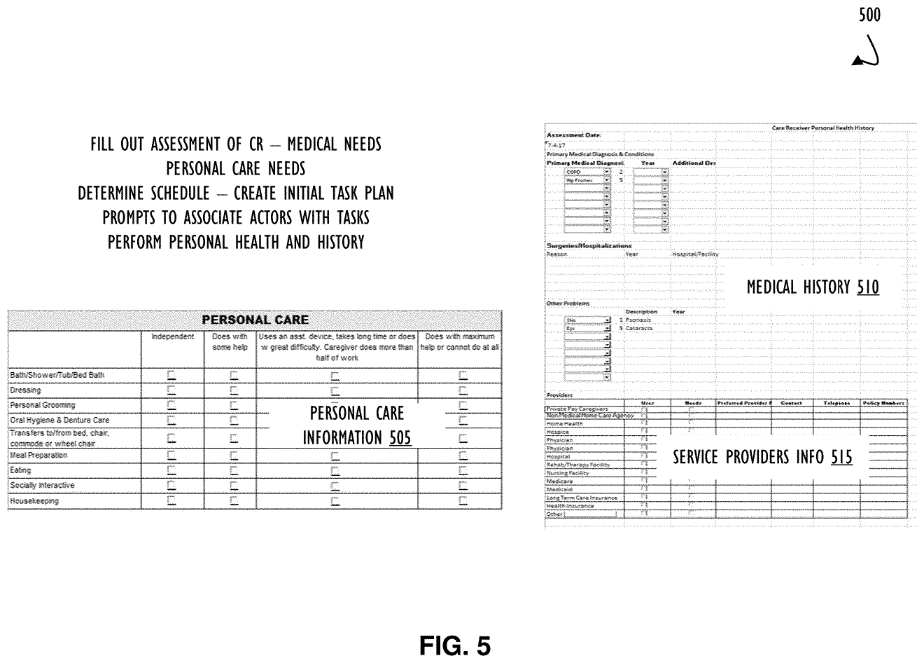

Referring now to FIG. 5, an example of a first set of tabular representations 500 of support data that may be managed within a care management system for a particular CR is illustrated. After completing the definitional phase discussed above for FIG. 4 (and in more detail below for FIG. 10), assessment information or support data may be gathered for the CR. This assessment information may include many different types of information. In this example, personal care information 505, medical history information 510, and service provider information 515 may be obtained. Obtaining of data may be through data entry by a CR (or other actor) or may be performed via data import from one or more external data sources.

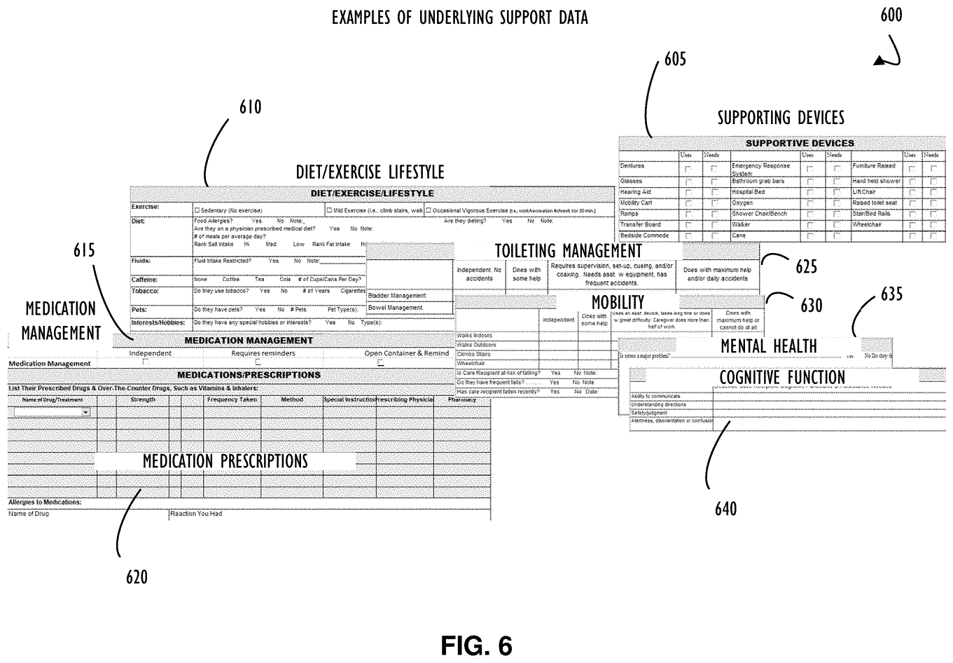

Referring now to FIG. 6, an example of a second set of tabular representations 600 of support data that may be managed within a care management system for a particular CR is illustrated. The types of data illustrated in FIG. 6 include data about supporting devices 605, diet and exercise 610 for a particular CR, medication management 615, medication prescriptions 620, toileting management 625, mobility 630, mental health 635, and cognitive functions 640. This information may change over time and the disclosed system may use the initially entered information as a baseline or initial condition to detect changes that may indicate further attention. Information other than those specifically show in FIGS. 5 and 6 may also be gathered.

As mentioned above, data may be imported from external sources either one time or periodically. One goal of the disclosed care management system is to provide a central repository for actors to be able to assess the real-time needs of the CR. Accordingly, accurate and current data for the home care management of the CR may be important.

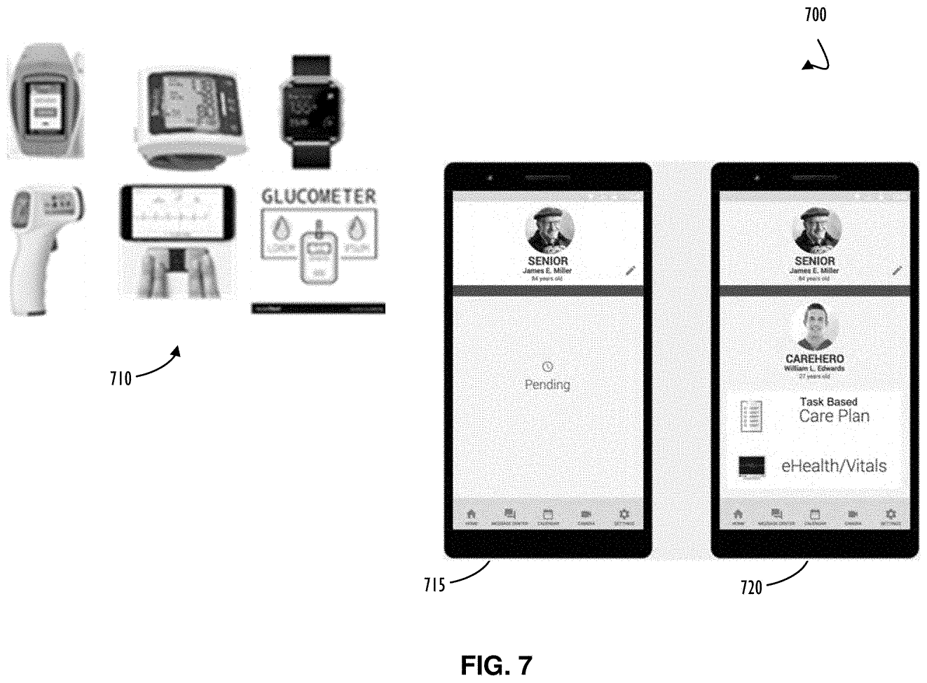

Referring now to FIG. 7, diagram 700 illustrates different devices that may interact with the disclosed care management system. Medical devices 710 may include any device that measures information about the CR and each of medical devices 710 may be network enabled to automatically interface and share information with the care management system. Some example devices include, but are not limited to: blood pressure monitor, thermometer, EKG—heart monitor, Constant Glucose Monitoring (CGM), glucometer, pulse oximeter, pulse rate monitor, respiration rate monitor, smart watch, pill dispenser, oxygen supplementation device, scale, pacemaker, home exercise equipment, thermostat, video conferencing devices, IP cameras, voice to text devices, content delivery devices, home automation devices, glass break detectors, door/window chimes, steps counter, fall detection devices, infrared movement devices, games/activities (to increase and monitor cognitive functions), wander management devices (GPS location devices), bed mats, CO monitors, and smoke detectors.

In cases where one of medical devices 710 is not network enabled, that device may have another type of interface to upload information to the care management system. In cases where no interface is available, the actor using the device may interface and provide information through a user interface of a mobile device as illustrated for mobile devices 715 and 720. Note that mobile device 720 illustrates an interface where both the CR and the actor are shown as well as a task of collecting “vitals” for the CR to be performed by that specific actor.

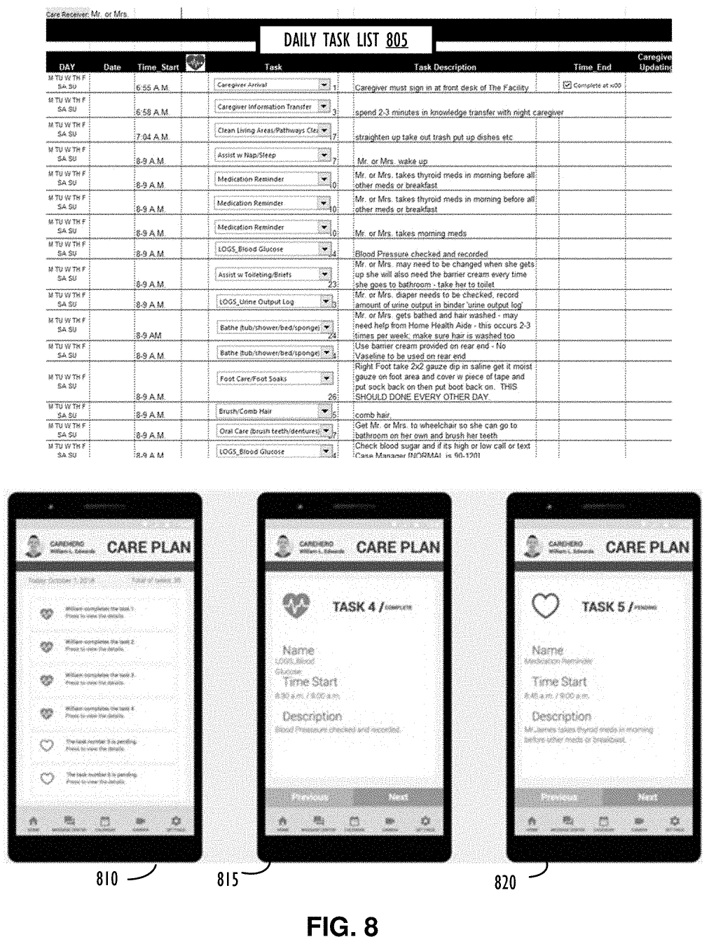

Referring now to FIG. 8, an example of a daily task list for a particular CR (i.e., in the form of a table) and how that daily task list may be presented on different end user devices for different actors as part of a care management system is illustrated. Daily task list 805, in this example, represents a “slice” of care plan 200 discussed above. The slice of the care plan provides a schedule of tasks that are to be completed near the current time of the current day. Because different tasks may be associated with (i.e., assigned to) different actors, example user interfaces are shown for mobile device 810 that shows a list of tasks, mobile device 815 which is isolated to task 4, and mobile device 820 which is isolated to task 5. In this manner, each task that is assigned to a particular actor may prompt that actor to perform that task via the user interface of the mobile device associated with an appropriate actor.

Once prompted to perform the task, user interface screens may collect information about the task and ensure that the task was completed. Once completed, the monitoring portion of the care management system may mark that task as completed and proceed to the next most important task. If a particular task is associated with a specific type of data collection, the user interface may ensure that the appropriate data has been made available prior to marking the task completed. Further, some task completions may result in an automatic notification being initiated to other actors within the system that are associated with the CR for which the task was completed.

In some cases, tasks may be associated with other tasks such that one task is a pre-requisite for the other task. Other types of task interaction are also possible. In any case, the care management system may be configured to provide linkages between tasks to ensure their completion in a proper order and to avoid prompting for a task that is not yet ready to be performed (i.e., missing a pre-requisite task). In cases where tasks are not being timely completed, escalation and notifications may be initiated automatically by the care management system to inform other actors that attention may be needed.

Referring now to FIG. 9, block diagram 900 is an example of actors and roles and their interaction possibilities with a particular CR utilizing the disclosed care management system. CR 905 is central in block diagram 900 and has associated care devices 906 that provide information to a care management system with a default that the information is about the directly associated CR 905. In some instances, it may be possible that a single medical device may be used for two different CRs (e.g., husband and wife both under home health care). Accordingly, the default association for a measurement from a given device may have an override for some implementations. As noted throughout this disclosure, a single actor may assume multiple roles within the disclosed care management system. It is possible for a doctor to also be a friend or even a family member.

Remote family 910 is a block at the top left of diagram 900 and illustrates that one or more actors may be defined as remote family 910 and have interactions with the care management system based on the role of “remote family member”. Local family 915 illustrates that one or more actors may be defined as local family. Accordingly, the local family role actors may have more immediate notifications and task assignments that are different from remote family. A local family member may perform different support tasks when compared to a remote family member and the disclosed care management system has logic to accommodate this distinction.

Care providers 920 illustrates that one or more actors may perform the role of a care provider. Care providers 920 may include doctors, nurses, therapists, care professionals, or others providing care to a CR. Care provider 920 may be assigned tasks from a care plan, may be provided alerts for measurement data from associated medical devices and may interact with the care management system to provide inputs relative to their care of the CR.

Home service providers 925 illustrate that activities managed by the disclosed home care management system are not strictly related to medical needs. For example, one goal of the disclosed system is to utilize an intelligent and functional “social network” of actors to assist a CR 905 in maintaining an established standard of living. This standard of living includes both a standard of medical care and a standard of living environment, such as a home or apartment that will need to be maintained. Home services 925 may include a handyman, cook, maid, butler, etc. that will provide a service other than medical care for the CR 905.

Medical services 930 illustrate that there may be a need to interface with a pharmacy or medical supply company to properly complete tasks within the care plan. Accordingly, the disclosed care management system may interface with those types of entities to place orders for supplies and medicine relative to directives provided within the specific care plan for the associated CR 905. Interactions may cause the disclosed system to correlate supplies with care plan tasks such that the supplies are available at the time when the task is scheduled.

Volunteers 935 may include local church members or friends of CR 905 who are willing to complete appropriate tasks from a care plan for CR 905. Tasks such as meal preparation may be performed by actors that are not specifically trained in medical procedures but are willing to help out. The disclosed care management system provides for that role and understands that help from others may be a welcome assistance to family members who are confronted with a long-term care of a particular CR 905.

Referring now to FIG. 10, example workflow 1000 for a definitional phase that represents a possible technique to provide definitional information for a particular CR to set up that CR within the disclosed home care management system. Workflow 1000 begins at block 1005, where initial patient data is obtained. Recall that a CR and a patient may refer interchangeably to a person that is being helped using the disclosed home care management system via an automated care plan. At block 1010, the name and personal information about the CR may be entered into the system or imported from some other source (e.g., hospital records). Block 1015 indicates that medical histories about comorbidities or other metrics of the CR may be entered or imported into the system.

Block 1020 indicates that actors (e.g., people performing actions to assist the CR and implement the care plan) may be identified, defined, and/or otherwise associated with a particular CR. Block 1025 indicates that roles may be established for each actor. An actor may have multiple roles within the system. Examples of roles could be family member, volunteer, medical professional, service provider, etc. Each of these roles could be further refined with additional attributes. Specifically, a family member may be designated as a “local” family member or a “remote” family member.

Accordingly, actions on a care plan that require local support may be assigned or performed by “local” family members as opposed to “remote” family members. Also, emergency notifications may first go to local family members prior to being sent to remote family members because a local family member may be better positioned to handle an “emergency” if one should arise. Further, the medical professional role may be further refined to identity the type of medical professional (e.g., doctor, nurse, therapist, etc.). In general, block 1025 indicates that associations between specific actors and the roles they will perform for the CR will be defined within the care management system.

Block 1030 indicates that roles may be used for data access criteria within the system. Specifically, roles may assist in conformity with the Health Information Portability and Privacy Act (“HIPPA”)—a statute about data access promulgated by the United States federal government. In the disclosed system, there may be a matrix of authorizations and security controls to determine which actors are allowed to view or alter different data parameters associated with the CR.

Block 1035 indicates that device associations may be defined. Specifically, a particular medical device with an automated information exchange interface (e.g., a network connected medical device) may be defined as providing information for a particular CR. Thus, when a temperature reading is obtained from a thermometer that is assigned to a CR the temperature reading will (by default) be associated within the system as that CR's current temperature measurement. Similarly, a blood pressure gauge, or a glucose monitor, which has been previously associated with a particular CR, would perform their measurement and automatically attempt to associate their readings with the CR. A pill dispenser may also be a device associated with the CR and automatically provide medications based on a schedule as prescribed for the CR.

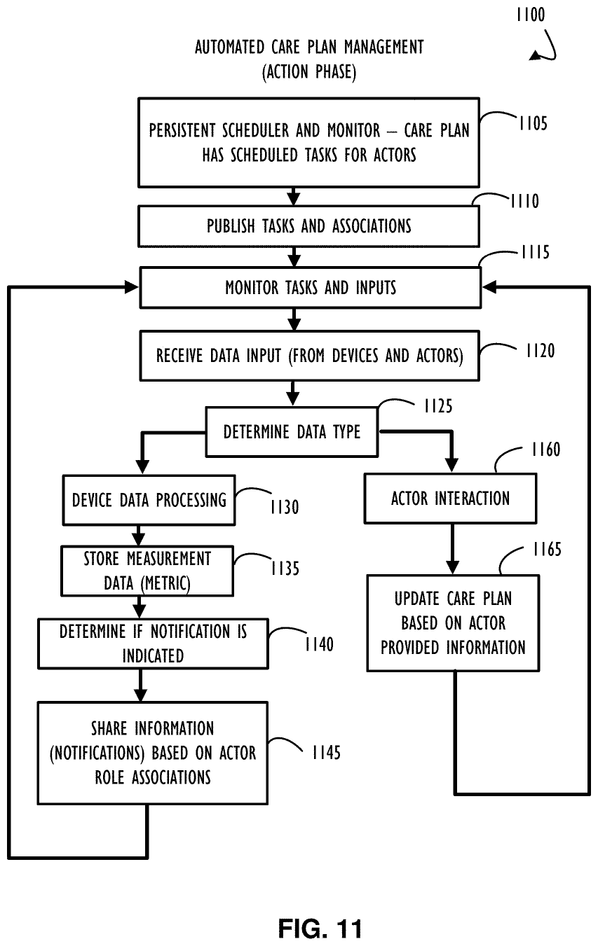

Block 1040 indicates that upon completion of the definitional phase an initial care plan may be created. Block 1045 indicates that the initial care plan may be validated by a medical professional (and family member) prior to initiating that care plan. Once validated and completed, block 1050 indicates that the care management system may proceed to the action phase for that care plan. One example action phase implementation within the care management system is discussed next with reference to FIG. 11.

Referring now to FIG. 11, one example workflow 1100 for an action phase of an automated care plan management as performed by logic of a computer system. The automated phase follows the definitional phase (described above relative to FIG. 10) and may be persistent throughout the care of a CR. Beginning at block 1105, persistent scheduler and monitor 1105 may execute on a computer system as a daemon or other persistent process. Periodically, as indicated at block 1110, tasks and associations for those tasks may be published such that all associated devices are either notified or aware of activities associated with a task of a care plan. Block 1115 indicates that the computer system may monitor for new tasks or other inputs associated with already completed (or in progress) tasks.

Upon receipt of an input at block 1115, flow continues to block 1120 to process the received data input. Block 1125 indicates that a data type for the received input may be determined and different action flows may be initiated based on the data type. For example, input from a device may proceed to block 1130 and, alternatively, input from a device associated with an actor may proceed to block 1160. Other types of data and other alternate data flows are also possible.

As mentioned above, block 1130 indicates that device data processing may be initiated. Block 1135 indicates that data from a medical device (i.e., a metric reading) may be stored and optionally processed. Block 1140 indicates that a determination about notification to a particular actor is warranted based on the metric. For example, if a low (or high) blood pressure reading is obtained, an automated notification may be sent to a particular actor (e.g., local family member).

Block 1145 indicates that notifications may be based on metric thresholds, actor role associations, and other information known about the actor and the severity of the metric (i.e., deviation from threshold). In one example, if an alert condition is detected, a local family member may be automatically notified whereas a remote family member may not get an alert (because there is nothing they can do immediately). Also, if the alert is not acknowledged within a specified period of time, the notification process can be expanded to include others (e.g., remote family) that may be able to actively find local assistance.

Referring back to block 1125, if the data type is based on actor interaction as indicated at block 1160, flow continues to block 1165 where the information provided as part of the actor interaction may be used to update the care plan. Although not shown in FIG. 11, it is possible that information from an actor interaction may also initiate some sort of alert processing similar to that described above.

As indicated in workflow 1100, flow consistently returns to block 1115 for persistent monitoring of tasks and inputs. Reminders of tasks that need to be performed based on the automated care plan may be sent in a similar manner to the automatic notifications discussed above. In general, the automated care plan drives the tasks that are monitored, and one or more actors complete those tasks to satisfy the care plan. Deviations or concerns about the care plan may result in notifications or other types of escalations in an attempt to ensure conformance with the care plan.

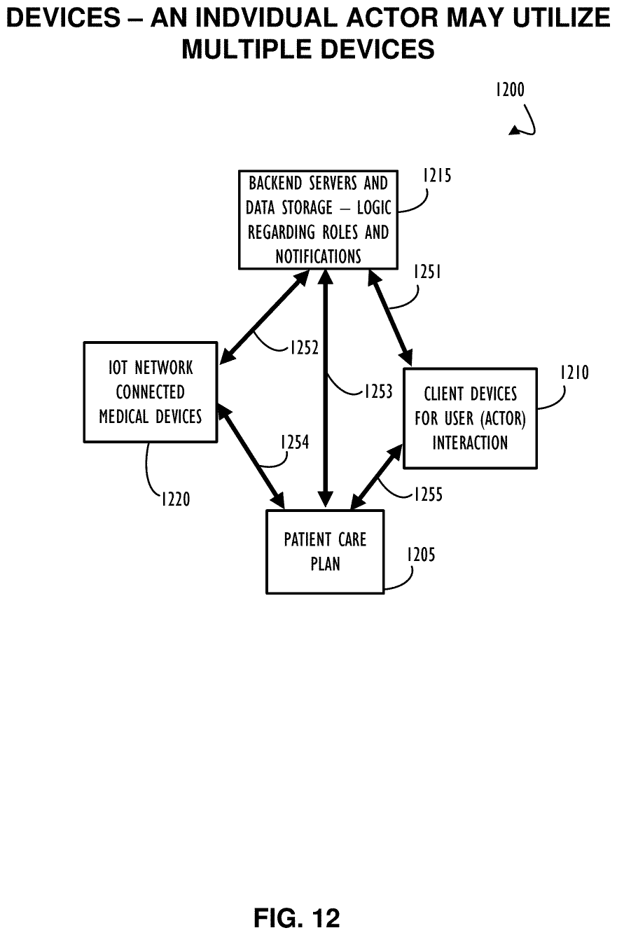

Referring now to FIGS. 12-15, examples of systems, system interactions, and system components that may be used to implement the disclosed home care management system cloud-based application will be discussed.

FIG. 12 illustrates example system interactions 1200 to collect and maintain information for use by a home care management system cloud-based application, in accordance with one or more examples of this disclosure. In FIG. 12, access interactions are indicated using a solid bidirectional arrow between interacting users and/or systems. For example, patient care plan 1205 represents a central repository for information regarding a particular CR. As illustrated by arrows 1253, 1254, and 1255, the patient care plan 1205 is a central interaction point for other systems. Note that patient care plan 1205 may not be an actual computer system but may be thought of as a logical repository of data that may be local to a single system or shared across multiple physical storage repositories.

In system interactions 1200, backend servers 1215 that may be cloud based can perform logic and collect data. As illustrated by arrow 1251, backend servers 1215 may obtain data from client devices 1210. Client devices 1210 may be associated with one or more actors and receive input (i.e., from an actor) regarding a care plan task. Specifically, an actor may be performing a care plan task or may be acknowledging that they are going to be committed to performing that task.

In system interactions 1200, IOT network connected medical devices 1220 may provide medical metric data and status updates related to a task for a CR (e.g., as indicated by arrows 1252 and 1254). For example, a blood pressure reading, a blood sugar reading, or some other type of medical metric may be obtained by the device and automatically integrated into the care management system information stores. The medical metric may be associated with a task in patient care plan 1205 and/or may be stored via backend servers 1215.

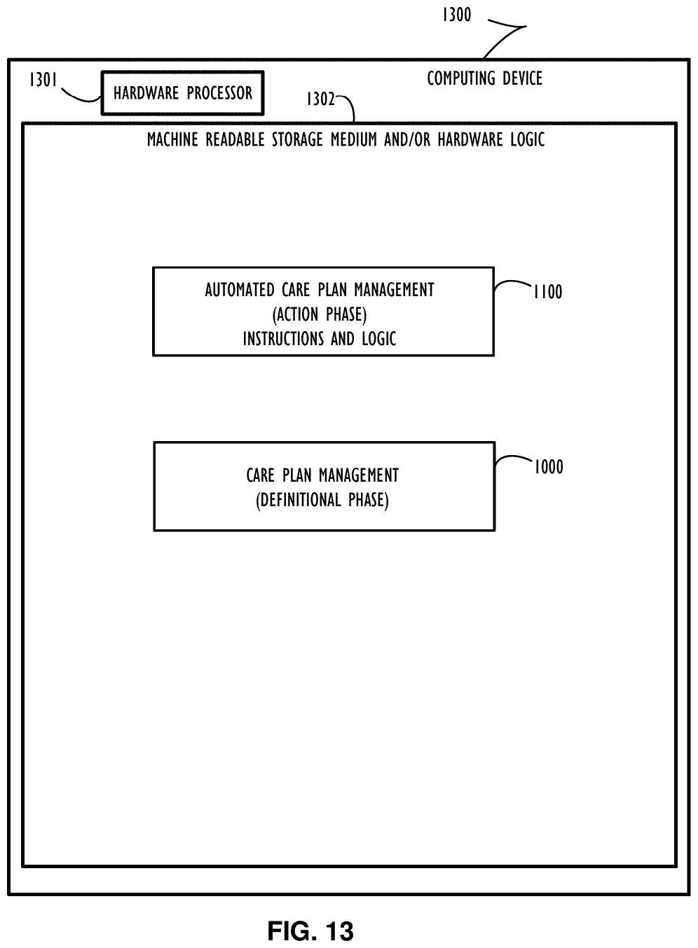

Referring now to FIG. 13, shown is an example computing device 1300, with a hardware processor 1301, and accessible machine-readable instructions stored on a machine-readable medium and/or hardware logic 1302 that may be used to perform one or more functions of the home care management system cloud-based application, according to one or more disclosed example implementations. Specifically, FIG. 13 illustrates computing device 1300 configured to perform the example workflows 1000 or 1100 as an example. However, computing device 1300 may also be configured to perform the flow of other methods, techniques, functions, or processes described in this disclosure. In this example of FIG. 13, machine-readable storage medium 1302 includes instructions to cause hardware processor 1301 to perform blocks 1005-1050 discussed above with reference to FIG. 10 and/or blocks 1105-1165 discussed above with reference to FIG. 11. Different implementations of workflows 1000 and 1100 are possible, including hardware logic configured on a chip to implement all or part of workflows 1000 and/or 1100 in conjunction with an overall implementation of disclosed techniques to provide a cloud-based home care management system application.

A machine-readable storage medium, such as 1302 of FIG. 13, may include both volatile and nonvolatile, removable and non-removable media, and may be any electronic, magnetic, optical, or other physical storage device that contains or stores executable instructions, data structures, program module, or other data accessible to a processor, for example firmware, erasable programmable read-only memory (“EPROM”), random access memory (“RAM”), non-volatile random access memory (“NVRAM”), optical disk, solid state drive (“SSD”), flash memory chips, and the like. The machine-readable storage medium may be a non-transitory storage medium, where the term “non-transitory” does not encompass transitory propagating signals.

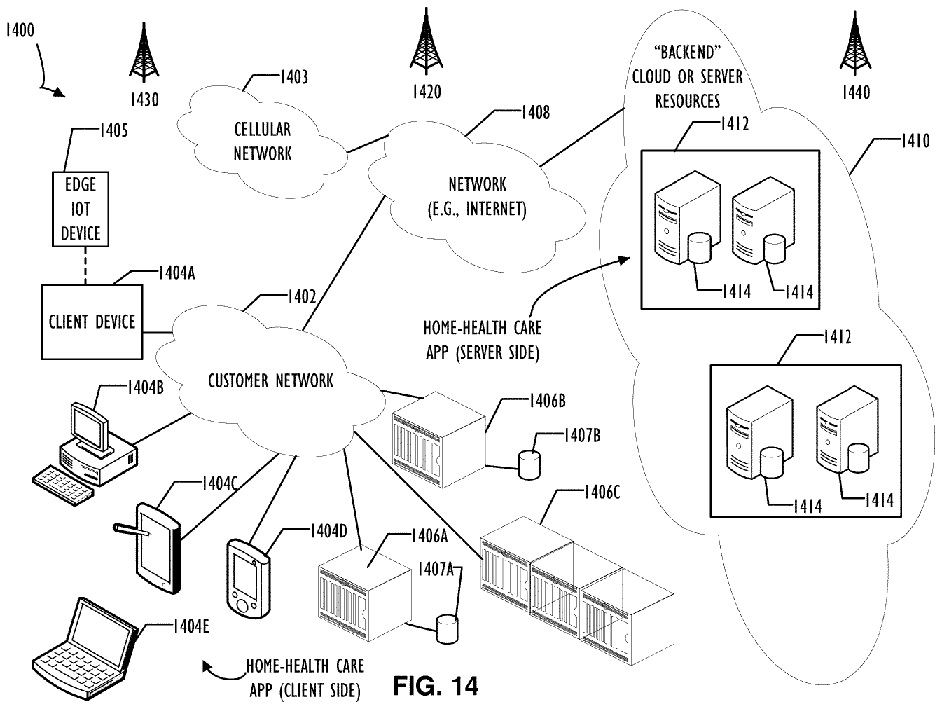

FIG. 14 represents a network infrastructure 1400 that may be used to implement all, or part of the disclosed cloud-based home care management application, according to one or more disclosed embodiments. Network infrastructure 1400 includes a set of networks where embodiments of the present disclosure may operate. Network infrastructure 1400 comprises a customer network 1402 (which may represent a home WiFi® network for a CR), network 1408 (which may be the Internet), cellular network 1403, and a cloud service provider network 1410. In one embodiment, customer network 1402 may be a local private network, such as local area network (“LAN”) that includes a variety of network devices that include, but are not limited to switches, servers, and routers.

Each of these networks can contain wired or wireless programmable devices and operate using any number of network protocols (e.g., Transmission Control Protocol/Internet Protocol, or “TCP/IP”) and connection technologies (e.g., WiFi® networks, or Bluetooth®. In another embodiment, customer network 1402 represents an enterprise network that could include or be communicatively coupled to one or more local area networks (“LANs”), virtual networks, data centers and/or other remote networks (e.g., 1408, 1410). In the context of the present disclosure, customer network 1402 may include one or more high-availability switches or network devices using methods and techniques such as those described above. Specifically, compute resource 14068 and/or compute resource 1406A may be configured as a network infrastructure device incorporating storage devices (e.g., 1407A and 1407B). An enterprise customer network may be utilized if a hospital were to implement one or more embodiments of the present disclosure to assist with multiple patients (“CRs”) at a single large facility rather than home-based care as discussed above.

As shown in FIG. 14, customer network 1402 may be connected to one or more client devices 1404A-E and allow the client devices 1404A-E to communicate with each other and/or with cloud service provider network 1410, via network 1408 (e.g., the Internet). Client devices 1404A-E may be computing systems such as desktop computer 1404B, tablet computer 1404C, mobile phone 1404D, laptop computer (shown as wireless) 1404E, and/or other types of computing systems generically shown as client device 1404A. In the examples of this disclosure, it is likely the different user types outlined in FIG. 12 may obtain access to the home care management system cloud-based application via a client device such as those illustrated in network infrastructure 1400.

Network infrastructure 1400 may also include other types of devices generally referred to as Internet of Things (“IOT”) (e.g., edge IOT device 1405) that may be configured to send and receive information via a network to access cloud computing services or interact with a remote web browser application (e.g., to receive information or respond to requested information). In some implementations edge IOT device 1405 may provide information to assist in automated task validation and/or automated medical metric gathering from a home-based medical device (e.g., such as those in FIG. 7). Specifically, if tasks are performed for a CR and information pertaining to that task is available to edge IOT device 1405 then that information may be uploaded to the disclosed home care management system cloud-based application. For example, a blood pressure gauge may incorporate edge IOT device 1405 and communicate that a blood pressure reading for a CR has been taken.

FIG. 14 also illustrates that customer network 1402 includes local compute resources 1406A-C that may include a server, access point, router, or other device configured to provide for local computational resources and/or facilitate communication amongst networks and devices. For example, local compute resources 1406A-C may be one or more physical local hardware devices. Local compute resources 1406A-C may also facilitate communication between other external applications, data sources (e.g., 1407A and 1407B), and services, and customer network 1402.

Network infrastructure 1400 also includes cellular network 1403 for use with mobile communication devices. Mobile cellular networks support mobile phones and many other types of mobile devices such as laptops etc. Mobile devices in network infrastructure 1400 are illustrated as mobile phone 1404D, laptop computer 1404E, and tablet computer 1404C. A mobile device such as mobile phone 1404D may interact with one or more mobile provider networks as the mobile device moves, typically interacting with a plurality of mobile network towers 1420, 1430, and 1440 for connecting to the cellular network 1403.

FIG. 14 illustrates that customer network 1402 is coupled to a network 1408. Network 1408 may include one or more computing networks available today, such as other LANs, wide area networks (“WAN”), the Internet, and/or other remote networks, in order to transfer data between client devices 1404A-D and cloud service provider network 1410 (e.g., a cloud service provider hosting the disclosed home care management system application). Each of the computing networks within network 1408 may contain wired and/or wireless programmable devices that operate in the electrical and/or optical domain.

In FIG. 14, cloud service provider network 1410 is illustrated as a remote network (e.g., a cloud network) that is able to communicate with client devices 1404A-E via customer network 1402 and network 1408. The cloud service provider network 1410 acts as a platform that provides additional computing resources to the client devices 1404A-E and/or customer network 1402. In one embodiment, cloud service provider network 1410 includes one or more data centers 1412 with one or more server instances 1414. Cloud service provider network 1410 may also include one or more frames or clusters (and cluster groups) representing a scalable compute resource that may implement the techniques of this disclosure.

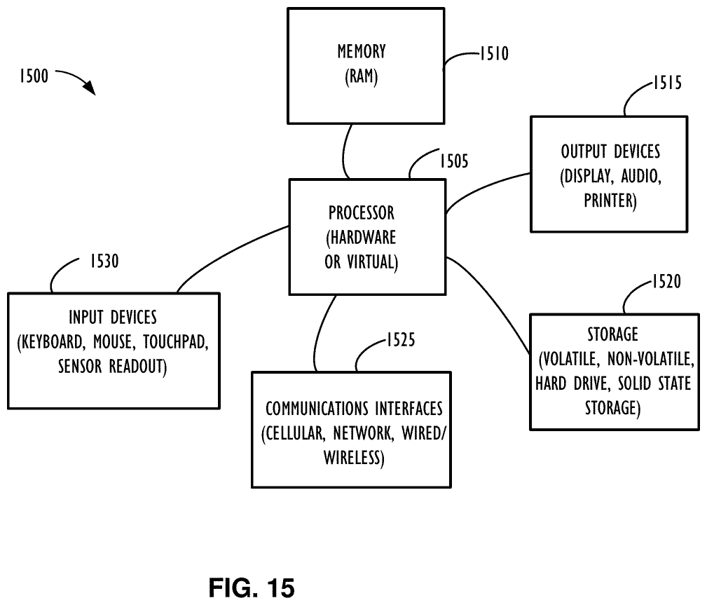

FIG. 15 illustrates a computing device 1500 that may be used to implement or be used with the functions, modules, processing platforms, execution platforms, communication devices, and other methods and processes of this disclosure. For example, computing device 1500 illustrated in FIG. 15 could represent a client device or a physical server device and include either hardware or virtual processor(s) depending on the level of abstraction of the computing device. In some instances (without abstraction), computing device 1500 and its elements, as shown in FIG. 15, each relate to physical hardware. Alternatively, in some instances one, more, or all of the elements could be implemented using emulators or virtual machines as levels of abstraction. In any case, no matter how many levels of abstraction away from the physical hardware, computing device 1500 at its lowest level may be implemented on physical hardware.

As also shown in FIG. 15, computing device 1500 may include one or more input devices 1530, such as a keyboard, mouse, touchpad, or sensor readout (e.g., biometric scanner) and one or more output devices 1515, such as displays, speakers for audio, or printers. Some devices may be configured as input/output devices also (e.g., a network interface or touchscreen display).

Computing device 1500 may also include communications interfaces 1525, such as a network communication unit that could include a wired communication component and/or a wireless communications component, which may be communicatively coupled to processor 1505. The network communication unit may utilize any of a variety of proprietary or standardized network protocols, such as Ethernet®, TCP/IP, to name a few of many protocols, to effect communications between devices. Network communication units may also comprise one or more transceiver(s) that utilize the Ethernet®, power line communication (“PLC”), WiFi®, cellular, and/or other communication methods.

As illustrated in FIG. 15, computing device 1500 includes a processing element such as processor 1505 that contains one or more hardware processors, where each hardware processor may have a single or multiple processor cores. As mentioned above, each of the multiple processor cores may be paired with a task scheduler to facilitate implementations of this disclosure. In one embodiment, the processor 1505 may include at least one shared cache that stores data (e.g., computing instructions) that are utilized by one or more other components of processor 1505. For example, the shared cache may be a locally cached data stored in a memory for faster access by components of the processing elements that make up processor 1505. In one or more embodiments, the shared cache may include one or more mid-level caches, such as level 2 (“L2”), level 3 (“L3”), level 4 (“L4”), or other levels of cache, a last level cache (“LLC”), or combinations thereof. Examples of processors include but are not limited to a central processing unit (“CPU”) a microprocessor. Although not illustrated in FIG. 15, the processing elements that make up processor 1505 may also include one or more of other types of hardware processing components, such as graphics processing units (“GPU”), application specific integrated circuits (“ASICs”), field-programmable gate arrays (“FPGAs”), and/or digital signal processors (“DSPs”).

FIG. 15 illustrates that memory 1510 may be operatively and communicatively coupled to processor 1505. Memory 1510 may be a non-transitory medium configured to store various types of data. For example, memory 1510 may include one or more storage devices 1520 that comprise a non-volatile storage device and/or volatile memory. Volatile memory, such as random-access memory (“RAM”), can be any suitable non-permanent storage device. The non-volatile storage devices 1520 can include one or more disk drives, optical drives, solid-state drives (“SSDs”), tap drives, flash memory, read only memory (“ROM”), and/or any other type of memory designed to maintain data for a duration of time after a power loss or shut down operation. In certain instances, the non-volatile storage devices 1520 may be used to store overflow data if allocated RAM is not large enough to hold all working data. The non-volatile storage devices 1520 may also be used to store programs that are loaded into the RAM when such programs are selected for execution.

Persons of ordinary skill in the art are aware that software programs may be developed, encoded, and compiled in a variety of computing languages for a variety of software platforms and/or operating systems and subsequently loaded and executed by processor 1505. In one embodiment, the compiling process of the software program may transform program code written in a programming language to another computer language such that the processor 1505 is able to execute the programming code. For example, the compiling process of the software program may generate an executable program that provides encoded instructions (e.g., machine code instructions) for processor 1505 to accomplish specific, non-generic, particular computing functions.

After the compiling process, the encoded instructions may then be loaded as computer executable instructions or process steps to processor 1505 from storage device 1520, from memory 1510, and/or embedded within processor 1505 (e.g., via a cache or on-board ROM). Processor 1505 may be configured to execute the stored instructions or process steps in order to perform instructions or process steps to transform the computing device into a non-generic, particular, specially programmed machine or apparatus. Stored data, e.g., data stored by a storage device 1520, may be accessed by processor 1505 during the execution of computer executable instructions or process steps to instruct one or more components within the computing device 1500.

A user interface (e.g., output devices 1515 and input devices 1530) can include a display, positional input device (such as a mouse, touchpad, touchscreen, or the like), keyboard, or other forms of user input and output devices. The user interface components may be communicatively coupled to processor 1505. When the output device is or includes a display, the display can be implemented in various ways, including by a liquid crystal display (“LCD”) or a cathode-ray tube (“CRT”) or light emitting diode (“LED”) display, such as an organic light emitting diode (“OLED”) display. Persons of ordinary skill in the art are aware that the computing device 1500 may comprise other components well known in the art, such as sensors, powers sources, and/or analog-to-digital converters, not explicitly shown in FIG. 15.

Certain terms have been used throughout this description and claims to refer to particular system components. As one skilled in the art will appreciate, different parties may refer to a component by different names. This document does not intend to distinguish between components that differ in name but not function. In this disclosure and claims, the terms “including” and “comprising” are used in an open-ended fashion, and thus should be interpreted to mean “including, but not limited to . . . .” Also, the term “couple” or “couples” is intended to mean either an indirect or direct wired or wireless connection. Thus, if a first device couples to a second device, that connection may be through a direct connection or through an indirect connection via other devices and connections. The recitation “based on” is intended to mean “based at least in part on.” Therefore, if X is based on Y, X may be a function of Y and any number of other factors.

The above discussion is meant to be illustrative of the principles and various implementations of the present disclosure. Numerous variations and modifications will become apparent to those skilled in the art once the above disclosure is fully appreciated. It is intended that the following claims be interpreted to embrace all such variations and modifications.

What is claimed is:

1. A computer-implemented method to automatically generate and maintain a care plan of tasks for a care recipient (“CR”), the method comprising:
obtaining a first set of medical information pertaining to the CR, the first set of medical information reflecting a medical history for the CR;
obtaining a second set of maintenance metrics reflecting a level of care to be provided for the CR;
associating one or more actors with the CR, the one or more actors to complete at least one task selected or assigned from the care plan of tasks;
analyzing, using a programmed computer processor, the first set of medical information and the second set of maintenance metrics;
generating the care plan of tasks based on the analyzing;
assigning a first one of the one or more actors to complete a first task from the generated care plan of tasks;
monitoring inputs, after assignment of the first task, to determine completion of the first task; and
automatically providing notification to an actor from the one or more actors, other than the first actor, as a result of the monitoring of inputs indicating that an assigned task has not been completed as scheduled.

2. The computer-implemented method of claim 1, wherein monitoring inputs includes monitoring for data received from a network attached medical device.

3. The computer-implemented method of claim 2, wherein the network attached medical device is an Internet of things (“IOT”) medical device.

4. The computer-implemented method of claim 2, wherein the network attached medical device includes one more of: a thermometer, a blood sugar gauge, a blood pressure gauge, a medicine dispenser, a smart watch, a heart monitor, and a patient monitor.

5. The computer-implemented method of claim 3, wherein the IOT medical device receives commands remotely input by a second one of the one or more actors.

6. The computer-implemented method of claim 1, further comprising:
automatically updating the care plan of tasks as a result of the monitoring of inputs indicating that an assigned task has been completed.

7. The computer-implemented method of claim 6, further comprising:
performing a second generating of the care plan of tasks at an end of a first day to reflect a new care plan of tasks for a next day.

8. The computer-implemented method of claim 1, wherein the automatically provided notification is filtered using a set of filters based on roles of the one or more actors and locations of the one or more actors such that only a subset of actors, as determined by the filters, is provided the notification.

9. A non-transitory computer-readable medium including instructions store thereon that when executed by a computer processor cause the computer processor to generate and maintain a care plan of tasks for a care recipient (“CR”), the instructions to cause the computer processor to:
obtain a first set of medical information pertaining to the CR, the first set of medical information reflecting a medical history for the CR;
obtain a second set of maintenance metrics reflecting a level of care to be provided for the CR;
associate one or more actors with the CR, the one or more actors to complete at least one task selected or assigned from the care plan of tasks;
analyze, using a programmed computer processor, the first set of medical information and the second set of maintenance metrics;
generate the care plan of tasks based on the analysis;
assign a first of the one or more actors to complete a first task from the generated care plan of tasks;
monitor inputs, after assignment of the first task, to determine completion of the first task; and
automatically provide notification to an actor from the one or more actors, other than the first actor, as a result of the monitoring of inputs indicating that an assigned task has not been completed as scheduled.

10. The non-transitory computer-readable medium of claim 9, wherein the instructions to monitor inputs include instructions to monitor for data received from a network attached medical device.

11. The non-transitory computer-readable medium of claim 10, wherein the network attached medical device is an Internet of things (“IOT”) medical device.

12. The non-transitory computer-readable medium of claim 10, wherein the network attached medical device is selected from the group consisting of: a thermometer, a blood sugar gauge, a blood pressure gauge, a medicine dispenser, a smart watch, a heart monitor, and a patient monitor.

13. The non-transitory computer-readable medium of claim 11, wherein the IOT medical device receives commands remotely input by a second of the one or more actors.

14. The non-transitory computer-readable medium of claim 9, further comprising instructions to cause the computer processor to:
automatically update the care plan of tasks as a result of the monitored inputs indicating that an assigned task has been completed.

15. The non-transitory computer-readable medium of claim 14, further comprising instructions to cause the computer processor to:
perform a second generating of the care plan of tasks at an end of a first day to reflect a new care plan of tasks for a next day.

16. The non-transitory computer-readable medium of claim 9, wherein the instructions to cause the computer processor to automatically provide the notification include instructions to filter the notification using a set of filters based on the roles of the one or more actors and the locations of the one or more actors such that only a subset of actors, as determined by the filters, is provided the notification.

17. A system of communicatively attached computers that collectively provide a collaborative network of information regarding a care task plan for a care recipient (“CR”), the system comprising:
a collaborative network processing engine, executing on the system of computers, to collect and maintain data pertaining to the care task plan for the CR in a central storage repository of metric data, historical data, and future planning data;
a plurality of network attached medical devices communicatively coupled to the collaborative network processing engine;
a plurality of user devices communicatively coupled to the collaborative network;
a plurality of actors, each of the actors associated with one or more roles and at least one of the plurality of user devices, the one or more roles indicating a type of interaction between a respective actor and the CR; and
a plurality of security or privacy levels associated with data items obtained via inputs from the plurality of actors, via a respective user device, or from the plurality of network attached medical devices;
wherein the collaborative network processing engine is programmed with instructions to cause the collaborative network processing engine to:
obtain a first set of medical information pertaining to the CR, the first set of medical information reflecting a medical history for the CR;
obtain a second set of maintenance metrics reflecting a level of care to be provided for the CR;
associate one or more actors with the CR, the one or more actors to complete at least one task selected or assigned from the care plan of tasks;
analyze, using a programmed computer processor, the first set of medical information and the second set of maintenance metrics;
generate the care plan of tasks based on the analysis;
assign a first of the one or more actors to complete a first task from the generated care plan of tasks;
monitor inputs, after assignment of the first task, to determine completion of the first task; and
automatically provide notification to an actor from the one or more actors, other than the first actor, as a result of the monitoring of inputs indicating that an assigned task has not been completed as scheduled.

18. The system of claim 17, further comprising instructions to cause the collaborative network processing engine to:
automatically update the care plan of tasks as a result of the monitored inputs indicating that an assigned task has been completed.

19. The system of claim 14, further comprising instructions to cause the collaborative network processing engine to:
perform a second generating of the care plan of tasks at an end of a first day to reflect a new care plan of tasks for a next day.

20. The system of claim 17, wherein the instructions to cause the collaborative network processing engine to automatically provide notification include instructions to filter the notification using a set of filters based on roles of the one or more actors and locations of the one or more actors such that only a subset of actors, as determined by the filters, is provided the notification.
